SEQUENCE LISTING (18 sequences)

SEQ ID NO: 1 - protein, 12 aa, Artificial Sequence
  Membrane translocating sequence
AAVLLPVLLAAP

SEQ ID NO: 2 - protein, 8 aa, Artificial Sequence
  Membrane translocating sequence
AAVLLPVL

SEQ ID NO: 3 - protein, 9 aa, Artificial Sequence
  Membrane translocating sequence
AAVLLPVLL

SEQ ID NO: 4 - protein, 10 aa, Artificial Sequence
  Membrane translocating sequence
AAVLLPVLLA

SEQ ID NO: 5 - protein, 11 aa, Artificial Sequence
  Membrane translocating sequence
AAVLLPVLLAA

SEQ ID NO: 6 - protein, 8 aa, Artificial Sequence
  Membrane translocating sequence
LPVLLAAP

SEQ ID NO: 7 - protein, 9 aa, Artificial Sequence
  Membrane translocating sequence
LLPVLLAAP

SEQ ID NO: 8 - protein, 10 aa, Artificial Sequence
  Membrane translocating sequence
VLLPVLLAAP

SEQ ID NO: 9 - protein, 11 aa, Artificial Sequence
  Membrane translocating sequence
AVLLPVLLAAP

SEQ ID NO: 10 - protein, 8 aa, Artificial Sequence
  Tag sequence
DYKDDDDK

SEQ ID NO: 11 - protein, 8 aa, Artificial Sequence
  Tag sequence
WSHPQFEK

SEQ ID NO: 12 - protein, 10 aa, Artificial Sequence
  Tag sequence
EQKLISEEDL

SEQ ID NO: 13 - protein, 15 aa, Artificial Sequence
  Tag sequence
KETAAAKFERQHMDS

SEQ ID NO: 14 - protein, 19 aa, Artificial Sequence
  Tag sequence
KDHLIHNVHKEFHAHAHNK

SEQ ID NO: 15 - protein, 22 aa, Artificial Sequence
  Tag sequence
DYKDHDGDYKDHDIDYKDDDDK

SEQ ID NO: 16 - protein, 26 aa, Artificial Sequence
  Tag sequence
KRRWKKNFIAVSAANRFKKISSSGAL

SEQ ID NO: 17 - protein, 38 aa, Artificial Sequence
  Tag sequence
MDEKTTGWRGGHVVEGLAGELEQLRARLEHHPQGQREP

SEQ ID NO: 18 - protein, 51 aa, Artificial Sequence
  Tag sequence
TNPGVSAWQVNTAYTAGQLVTYNGKTYKCLQPHTSLAGWEPSNVPALWQLQ

(19) United States
(12) Patent Application Publication
(10) Pub. No.: US 2005/0032173 A1
(43) Pub. Date: Feb. 10, 2005

(54) Fusion proteins with a membrane translocating sequence and methods of using same to inhibit an immune response

(76) Inventors: Mauricio Rojas, Atlanta, GA (US); Ana L. Mora, Atlanta, GA (US)

Correspondence Address: TIM TINGKANG XIA;MORRIS, MANNING & MARTIN, LLP, 1600 ATLANTA FINANCIAL CENTER, 3343 PEACHTREE ROAD, N.E., ATLANTA, GA, 30326-1044, US

(21) Appl. No.: 10/634,645

(22) Filed: Aug. 5, 2003

(51) Int. Cl. C07K 14/47; C12Q 1/68; C07H 21/04; C12P 21/04

(52) U.S. Cl. 435/69.7; 435/320.1; 435/325; 530/350; 536/23.5; 435/6

(57) ABSTRACT

An isolated fusion protein. In one embodiment of the present invention, the isolated fusion protein includes a membrane-translocating peptide sequence of about 8 to about 50 residues comprising at least eight consecutive residues of SEQ ID NO: 1 (Ala-Ala-Val-Leu-Leu-Pro-Val-Leu-Leu-Ala-Ala-Pro), and an inhibitory IκB protein. Alternatively, the membrane-translocating sequence can have at least 9, 10, 11 or 12 twelve consecutive residues of SEQ ID NO: 1. The isolated infusion protein can be used to treat or prevent an immune response associated with an immune disorder or a disease or disorder related to apoptosis, such as cancer, in a host.

### FIELD OF THE INVENTION

This invention relates generally to fusion proteins with membrane translocating potential that can enter a cell and regulate gene expression to prevent or treat an immune response or a disease related to apoptosis in a host and methods of using same to inhibit such a response.

### BACKGROUND OF THE INVENTION

Discrimination between self and non-self antigens is the primary function of the immune system. Recognition of an antigen by the T cell receptor (TCR), activates a number of pathways that transmit signal from the cell surface into the nucleus. Studies from several groups suggest that one of the main pathways activated after TCR engagement is the NF-κB/Rel cascade (Ghosh et al., 1998; Li and Verma, 2002). Nuclear Factor K B (NF-κB) is a family of transcription factors that includes p50, p52, c-Rel, RelB and p65 (Rel A). In a resting state, NF-κB proteins are localized to the cytoplasm as homo or heterodimers. The most common of the NF-κB complex is the heterodimer p50/p65. In quiescent cells, NF-κB dimers are associated with certain inhibitory proteins called IκB proteins, specifically IκBα, IκBβ and IκBεε (Verma et al., 1995; Baldwin, 1996). IκBα may be the best-characterized IκB molecule. It contains serines at positions 32 and 36 that are phosphorylated by the activation complex of IκB kinases or IKK, inducing ubiquitination and proteosome mediated degradation of the IκBα molecule. After the IκBα protein is removed from the complex, NF-κB dimers are translocated into the nucleus, by exposure of a nuclear localization sequence (Brockman et al., 1995; Finco and Baldwin, 1995; Karin, 1999). Once in the nucleus NF-κB binds to specific DNA sequences and modulates gene expression. NF-κB promotes the expression of over 150 target genes. The large majority of proteins encoded by these genes participate in the immune responses including, cytokines, various immune-specific receptors, adhesion molecules and adaptor proteins (Baldwin, 2001).

The NF-κB family of transcription factors has also been demonstrated to play a role in regulating other cellular processes such as apoptotic cell death. These transcription factors can act as inducers or blockers of apoptosis in a stimulus- and cell type-dependent fashion. Studies have shown that these factors play a role in the embryonic responses to teratogens (Torchinsky et al., 2002).

Numerous efforts have been made to develop regulators of NF-κB activity (Epinat and Gilmore, 1999; Pahl, 1999; Yamamoto and Gaynor, 2001). The NF-κB cascade provides several targets where activation may be blocked: 1) blocking incoming signal pathways that activate the IKK complex; 2) interfering in the phosphorylation, ubiquitination and degradation of IκB proteins and 3) blocking the translocation of NF-κB dimers into the nucleus by targeting the nuclear pore protein complex used to import proteins into the nucleus. However, only methods that target the IκB molecules are specific for the NF-κB pathway.

In order to affect intracellular cell signaling, inhibitors must access the cell interior. Lin et al. (1995) have described a method of using a naturally-occurring signal peptide sequence to import a cargo peptide into a cell. U.S. Pat. Nos. 6,432,680 and 6,248,558 issued to Lin et al. disclose a non-naturally occurring 12 amino acid residue long membrane-translocating sequence (MTS) that can mediate the transport of peptides and even a full-length protein through the cell membrane into the cytoplasm. The MTS was originally derived from the hydrophobic region of the signal peptide of Kaposi Fibroblast Growth Factor (K-FGF) that has been modified to function as a cellular import signal. When an MTS motif is fused to the N+ or C− terminus of a protein, the fusion protein becomes membrane permeable (see for example, Lin et al., 1995; Lin et al., 1996; Liu et al., 1996; Rojas et al., 1996; Rojas et al., 1997; Du et al., 1998; Rojas et al., 1998). The MTS sequence can efficiently deliver up to several million molecules per cell (Fernandez and Bayley, 1998) of a large variety of proteins as fusion proteins. Successful delivery has been accomplished in many cell types and always is associated with preservation of protein function. Immunofluorescence and confocal imaging studies indicate the proteins are evenly distributed and stable in the cell (Du et al., 1998; Jo et al., 2001) and little or no cytotoxicity is generally seen.

Previous studies using the transgenic expression of an N-terminal portion of IκBα, IκBα-(ΔN) inhibitor in T cells have demonstrated the critical role of NF-κB in T cell development and the regulation of T cell proliferative and apoptotic responses (Boothby et al., 1997; Mora et al., 1999; Mora et al., 2001a; Mora et al., 2001b). Additionally, studies of allergic pulmonary inflammation using IκBα-(ΔN) transgenic mice have demonstrated that inhibition of NF-κB signal preferentially impairs type 1 as compared with type 2 T cell-dependent responses (Aronica et al., 1999). The defect in type 1 responses by inhibition of NF-κB also leads to a decreased incidence and severity of disease in the autoimmune model of collagen induced arthritis in mice (Seetharaman et al., 1999). Together these studies indicate that inhibition of NF-κB activation may be able to prevent T cell dependent autoimmune disease, although the transgene approach remains of limited clinical utility.

Therefore, a heretofore unaddressed need exists in the art to address the aforementioned deficiencies and inadequacies.

### SUMMARY OF THE INVENTION

Embodiments of the present invention provide fusion proteins that include a membrane-translocating peptide and methods of using same for preventing immune responses including a method for specifically inhibiting the NF-κB cascade within a cell in order to prevent or treat an immune response in a host.

In one aspect, the invention is related to an isolated fusion protein. In one embodiment of the present invention, the isolated fusion protein includes a membrane-translocating peptide sequence of about 8 to about 50 residues comprising at least eight consecutive residues of SEQ ID NO: 1 (Ala-Ala-Val-Leu-Leu-Pro-Val-Leu-Leu-Ala-Ala-Pro), and an inhibitory IκB protein. In other embodiments of the present invention, the membrane-translocating sequence can alternatively comprise at least 9, 10, 11 or 12 twelve consecutive residues of SEQ ID NO: 1. The IκB protein can be, in alternative embodiments of the invention, an IκBα protein, an IκBβ protein or an IκBε protein. The IκB protein can also be a complex formed by two or more IκB proteins. In any embodiment of the present invention, the fusion protein can be optionally attached to a tag amino acid sequence or protein.

In another aspect, the invention is related to a pharmaceutical composition that includes an isolated fusion protein and a pharmaceutically acceptable carrier. In one embodiment, the isolated fusion protein has a membrane-translocating sequence comprising at least eight consecutive residues of SEQ ID NO: 1 (Ala-Ala-Val-Leu-Leu-Pro-Val-Leu-Leu-Ala-Ala-Pro), and an IκB protein.

In a further aspect, the invention is related to methods for preventing, or alternatively, treating an immune response in a host by administering the isolated fusion protein. The immune response can be associated with at least one of an allergy, asthma, contact dermatitis, delayed-type hypersensitivity, a wound-healing, allergic rhinitis, food hypersensitivity, ectopic dermatitis, inflammatory bowel disease, an immunologic disease of the lung, eosinophilic pneumonias, idiopathic pulmonary fibrosis, hypersensitivity pneumonitis, an autoimmune or immune-mediated skin disease, a bullous skin disease, erythemia multiforme, psoriasis, gluten-sensitive enteropathy, Whipple's disease, systemic lupus erythematisis, rheumatoid arthritis, osteoarthritis, juvenile chronic arthritis, ankylosing spondlylitis, systemic sclerosis, an idiopathic inflammatory myopathy, Sjögren's disease, pleuritis, sarcoidosis, amyloidosis, autoimmune hemolytic anemia, autoimmune thrombocytopenia, thyroiditis, diabetes mellitus, immune-mediated renal disease, myasthenia gravis, a demylenating disease of the central or peripheral nervous system, idiopathic demylenating polyneuropathy, Guillain-Barre syndrome, a chonic inflammatory demyelinating polyneuropathy, a hepotobiliary disease, an infectious or autoimmune chronic active hepatitis, primary biliary cirrhosis, granulomatous hepatitis, sclerosing cholangitis, Graves disease, a transplantation-associated disease, a graft rejection, and a graft-versus-host disease. Furthermore, the immune response can be the result of exposure of the host to a pulmonary infectious agent such as Mycoplasma tuberculosis.

In another aspect, the present invention relates to a method of treating or preventing a disease or disorder related to apoptosis by administering the isolated fusion protein.

In still other embodiments of the present invention, the fusion protein is administered in combination with a compound or drug used to treat or prevent any immune-related disorder. In various embodiments, the compound can be an anti-inflammatory agent, an immunosuppressive agent, an antihistamine agent, or any pharmaceutically acceptable prodrug or derivative thereof.

In still another aspect, the present invention relates to an animal model that can be used to test the effects of an isolated fusion protein on an inflammatory response. An animal host is first injected with the test fusion protein. The animal host can be typically selected from a line of animals that express a reporter gene whose expression is mediated by a NF-κB-dependent process. Thus, inflammation or the inflammatory process will induce the production of the reporter gene product. Following injection of the test fusion product, inflammation can be stimulated through a suitable process such as injection of a stimulatory substance (e.g. cytokine) or the creation of a wound under appropriate circumstances. The reporter gene product can then be visualized and quantified in the test subject and then compared with the amount of reporter gene product in appropriately treated control animal.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 depicts the expression, purification, and preservation of antigenicity of the recombinant proteins glutathione S-transferase (GST)-IκBα-(ΔN) and GST-IκBα-(ΔN)-MTS. (A) shows biomass obtained from a bacterial culture that was lysed by enzymatic digestion, and a glutathione agarose beads column purified fusion proteins. Panel (B) depicts purified recombinant GST-IκBα-(ΔN) and GST-IκBα-(ΔN)-MTS that were subjected to SDS-PAGE.

FIG. 2 shows that imported MTS fusion proteins localize to the cytoplasm in cells incubated with GST-IκBα-(ΔN) or GST-IκBα-(ΔN)-MTS proteins.

FIG. 3 shows that the nuclear translocation of NF-κB complexes was detected only in treated cells or cells pre-treated with IκBα-(ΔN).

FIG. 4 depicts the inhibition of nuclear translocation of NF-κB by IκBα-(ΔN)-MTS in primary thymocytes is specific to the permeable inhibitors and is dose-dependent.

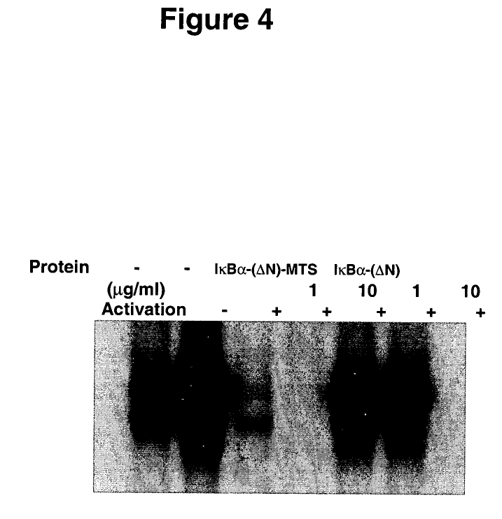

FIG. 5 schematically shows the inhibition in DNA synthesis in CD4+ T cells after T cell receptor stimulation by the permeable inhibitor IκBα-(ΔN)-MTS.

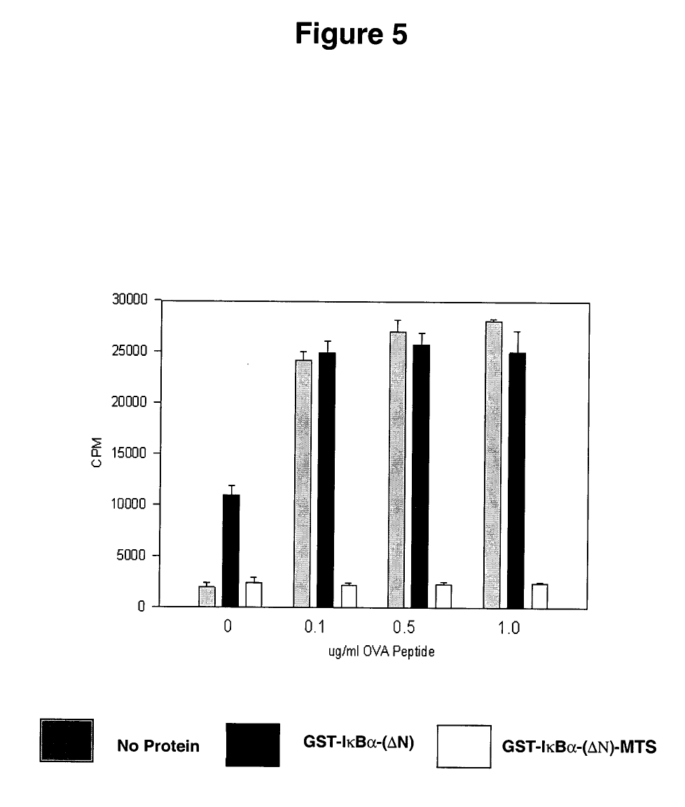

FIG. 6 depicts that systemic administration of the permeable inhibitor IκBα-(ΔN)-MTS to a mouse host reduced NF-κB activity induced by a local inflammatory process.

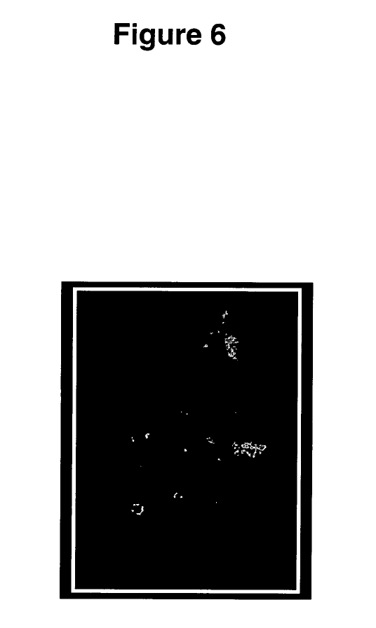

FIG. 7 is a photomicrograph of a sheep lung showing the distribution of labeled GST-IκBα-(ΔN)-MTS following aerosol administration.

### DETAILED DESCRIPTION OF THE PREFERRED EMBODIMENTS

Various embodiments of the invention are now described in detail. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner in describing the compositions and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

In order to manipulate intracellular processes mediated by endogenous proteins, regulatory molecules must access the cell interior, without altering cell function. Several efforts have attempted to deliver proteins to cryptic biologic spaces by delivering the appropriate gene. Much of this work has focused on delivery of a gene encoding wild-type protein into the lung using either viral or non-viral vectors. Although this technology could theoretically be used to deliver genes encoding proteins that would alter transcription factor activation or effect, current technologies, both viral and non-viral, have been disappointing (Marshall, 2002b; Marshall, 2002a; Kaiser, 2003; Marshall, 2003).

MTS technology has been reported as a method to produce proteins that have inherent cell-membrane translocating activity (Rojas et al., 1996; Rojas et al., 1997; Rojas et al., 1998). MTS fusion proteins are translocated with high efficiency into a large variety of cells including endothelial and epithelial cells, by a process that depends on time, temperature and protein concentration.

This highly efficient delivery system can be used to study and manipulate various intracellular processes. For example, a cell-permeable form of the EGF-receptor binding protein Grb2-SH2 coupled to MTS through the binding protein has been used to study EGF-induced mitogenic pathways (Rojas et al., 1996). Zhao et al demonstrated the versatility and efficacy of the MTS system by delivering a functional antibody into NIH3T3 cells (Zhao et al., 2001). Wang and Wang, using MTS technology, demonstrated that it is possible with a single immunization to enhance the CD4⁺ and CD8⁺ immune responses against some specific tumors resulting in complete inhibition of lung metastases and inducing protection from future tumor challenge (Wang et al., 1999). In addition, MTS technology has been utilized to delivery adaptor proteins, enzymes, transcription factors and kinases, in order to clarify the mechanisms of cell transformation, oncogenesis and cardiac hypertrophy (Turkson et al., 2001; Wu et al., 2002).

Among other things, the current invention can be practiced to provide the intracellular delivery of the NF-κB inhibitor, IκBα-(ΔN). The protein delivered is effective in cell lines, primary cells and in a live animal model. Specifically, the invention relates generally to methods for preventing immune responses by the administration of fusion proteins that include a membrane-translocating peptide. In one aspect, the invention relates to a method for preventing an immune response in a host by administering an isolated fusion protein. The fusion protein is made up of a membrane-translocating peptide and an inhibitory IκB protein. Another aspect of the invention relates to a method for treating an immune disorder in a host by administering the fusion protein. The invention further relates to an animal model that can be used to test the effects of a fusion protein coupled to a membrane-translocating peptide on the development of an inflammatory immune response.

Definitions

As used herein, the term “peptide” is intended to include mimetics and the term “amino acid” is intended to include D-form amino acids and modified amino acids. These substitutions may be made by someone of skill in the art, using the known structural similarities between the molecules. The term “polypeptide” refers to a linear polymer of amino acids linked via peptide bonds. Generally a polymer of relatively few amino acids is referred to as a “peptide” while a “polypeptide” may contain several, up to about 50-100, amino acids in a single strand. “Protein” refers to a large molecule composed of one or more polypeptide chains arranged in a 3-dimensional structure.

“Isolated protein” as disclosed herein, means any protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes.

The term “tag protein” is meant to designate any one or more peptides, polypeptides or proteins known to those skilled in the art that are attached to either the N-terminal or C-terminal portion or both, of the fusion protein. Such attachment can include, but is not limited to, a covalent bond between the tag protein and the fusion protein.

“Fusion protein” or “MTS-IκB fusion protein” a “MTS-IκBα” as disclosed herein, means any protein that includes a membrane translocating sequence (MTS) as disclosed herein, attached to any inhibitory IκB protein or combination of IκB proteins as disclosed herein. “IκB protein” is meant to include any individual IκB protein or combination of IκB proteins. It is also understood that the fusion protein of the present invention can include multimers (e.g., heterodimers or homodimers) or complexes with itself or other proteins. As a result, pharmaceutical compositions as described herein may comprise a protein of the invention in such multimeric or complexed forms. The membrane translocating sequence as disclosed herein may be located immediately adjacent to, or some distance from, the IκB protein as disclosed herein. Therefore, it is also understood that the by “fusion protein” is also intended to include any peptide or protein tag sequence either N-terminal or C-terminal to the listed sequence, or both.

The term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

“Treatment” includes the application or administration of a therapeutic agent to a subject or to an isolated tissue or cell line from a subject, who is afflicted with a disease, a symptom of disease or a predisposition toward a disease, with the goal of curing, healing, alleviating, relieving, altering, remedying, ameliorating, improving or affecting the disease, the symptoms of disease or the predisposition toward disease. “Treatment” refers to both therapeutic treatment and prophylactic or preventative measures, wherein the object is to prevent or slow down (lessen) the targeted condition or disorder. Those in need of treatment include those already with the disorder as well as those prone to have the disorder or those in whom the disorder is to be prevented.

“Chronic” administration refers to administration of fusion protein or pharmaceutical compositions in a continuous mode as opposed to an acute mode, so as to maintain the initial therapeutic effect (activity) for an extended period of time.

“Intermittent” administration is treatment that is not consecutively done without interruption, but rather is cyclic in nature.

“Mammal” or “host” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal or host is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to mammal or host being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins: hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term “immune-related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

The term “effective amount” is a concentration or amount of the isolated fusion protein or an accompanying drug or agent that results in achieving a particular stated purpose. An “effective amount” may be determined empirically. Furthermore, a “therapeutically effective amount” is a concentration or amount of the isolated fusion protein or accompanying drug, which is effective for achieving a stated therapeutic effect. This amount may also be determined empirically.

Construction & Design of the IκB-Membrane-Translocating Fusion Protein Membrane-Translocating Protein Sequence

The present invention relates to a method of preventing an immune or an inflammatory response by administering a membrane-translocating peptide coupled to an IκB protein in order to inhibit the NF-κB intracellular cascade.

The isolated fusion protein of the present invention comprises an inhibitory IκB protein coupled to an artificial membrane translocation sequence (MTS). The MTS consists of a peptide sequence of 8 to 12 amino acids. Such a peptide is described in U.S. Pat. No. 6,432,680 (Lin et al.), incorporated herein by reference. In one embodiment of the present invention, the amino acid sequence of the 12-residue membrane-translocating peptide is Ala-Ala-Val-Leu-Leu-Pro-Val-Leu-Leu-Ala-Ala-Pro (SEQ ID NO: 1).

The amino acid sequence is also intended to include an MTS comprising fewer than twelve residues, as signal peptide sequences of as few as eight amino acids provide membrane translocation of peptides across membranes within the cell. Thus, in various embodiments of the present invention, alternative MTS sequences can be comprised of amino acid sequence of eight (8) to twelve (12) consecutive amino acids chosen from SEQ. ID NO. 1. Exemplary of such alternative MTS sequences are Ala-Ala-Val-Leu-Leu-Pro-Val-Leu (SEQ. ID NO. 2), Ala-Ala-Val-Leu-Leu-Pro-Val-Leu-Leu (SEQ. ID NO. 3), Ala-Ala-Val-Leu-Leu-Pro-Val-Leu-Leu-Ala (SEQ. ID NO. 4), Ala-Ala-Val-Leu-Leu-Pro-Val-Leu-Leu-Ala-Ala (SEQ. ID NO. 5), Leu-Pro-Val-Leu-Leu-Ala-Ala-Pro (SEQ. ID NO. 6), Leu-Leu-Pro-Val-Leu-Leu-Ala-Ala-Pro (SEQ. ID NO. 7), Val-Leu-Leu-Pro-Val-Leu-Leu-Ala-Ala-Pro (SEQ. ID NO. 8), and Ala-Val-Leu-Leu-Pro-Val-Leu-Leu-Ala-Ala-Pro (SEQ. ID NO. 9). Alternative MTS sequences are intended to include alternative amino acids, as well as additional C-terminal or N-terminal amino acids as described for SEQ. ID. NO. 1. It is to be understood that amino acid sequences may include additional residues, particularly N- or C-terminal amino acids and still be essentially as set forth in the sequences disclosed herein, as long as the sequence confers membrane permeability upon the protein moiety of the fusion protein.

The MTS can be constructed by conventional modes of peptide synthesis well known to those skilled in the art. Alternatively, one skilled in the art would easily recognize that genetic engineering techniques could also be used to construct the MTS. As a non-limiting example, a DNA sequence encoding the 12-amino acid peptide is utilized to construct an MTS plasmid expression vector which then is incorporated with the sequences for the IκB protein. The vector expresses the fusion protein which can then be purified for import into the cell.

The fusion protein of the present invention incorporates a cleavage site located between the MTS and the IκB protein. This site may alternatively be any site that is known to those of skilled in the art to affect the cleavage of the fusion protein to physically remove the MTS from the IκB protein.

IκB Target Protein

Recognition of an antigen by the T cell receptor (TCR), activates a number of pathways that transmit signal from the cell surface into the nucleus. One of the main pathways activated after TCR engagement is the NF-κB/Rel cascade (Ghosh et al., 1998; Li and Verma, 2002). Nuclear Factor KB (NF-κB) is a family of transcription factors that includes p50, p52, c-Rel, RelB and p65 (Rel A). In a resting state NF-κB proteins are localized to the cytoplasm as homo or heterodimers; the most common of NF-κB complex is the heterodimer p50/p65. In quiescent cells, NF-κB dimers are associated with IκB inhibitory proteins (IκBα, IκBβ, IκBεε; Verma et al., 1995; Baldwin, 1996).

Generally, the active transcription factor NF-κB is translocated to the nucleus of the cell and can stimulate an inflammatory response. Unphosphorylated Iκββ complexes with NF-κB to inhibit the translocation and thus prevent the inflammatory process. However, phosphorylated IκB cannot complex with NF-κB. Thus, in the embodiments of the present invention, MTS-IκB is transduced into the cells of the host. The MTS-IκB is unphosporylated and therefore increases the pool of unphosphorylated IκB in the cell. The MTS-IκB may bind to NF-κB in the cell and prevent or reduce the inflammatory process or immune response by inhibiting NF-κB's nuclear translocation.

IκB proteins can be purified by a variety of methods known to those skilled in the art. As a non-limiting example, Craig, et al. (U.S. Pat. No. 5,004,688, incorporated herein by reference) describes purification of heterologous protein produced in Pichia. Techniques for protein purification from yeast expression systems are also well known to those of skilled in the art. In the Pichia system, commercially available vectors can be chosen from among those that are more suited for the production of cytosolic, non-glycosylated proteins and those that are more suited for the production of secreted, glycosylated proteins, or those directed to an intracellular organelle, so that appropriate protein expression can be optimized for the target protein of choice.

Alternatively, the inhibitory IκB protein may be recovered from culture medium or from host cell lysates. If membrane-bound, it can be released from the membrane using a suitable detergent solution (e.g. Triton-X 100) or by enzymatic cleavage. Cells employed in expression of IκB proteins can be disrupted by various physical or chemical means, such as freeze-thaw cycling, sonication, mechanical disruption, or cell lysing agents.

Furthermore, it may be desirable to purify an IκB protein from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the IκB protein. Various methods of protein purification may be employed and such methods are known in the art and described for example in Deutscher, 1982 and Scopes, et al. (1982). The purification step(s) selected will depend on a variety of parameters including, but not limited to, the nature of the production process used and the particular IκB protein produced.

Attachment of The MTS to the IκB Protein

The IκB protein can be covalently attached to the MTS by any of a variety of means that would be known to those skilled in the art. As a non-limiting example, orthogonal coupling methods for peptides and polypeptides involving a thioester intermediate have been described by Tam, et al. (1995). An MTS as described herein can be attached to an IκB target protein using methods such as those described by Tam et al.

Attachment of the Fusion Protein to Tag Proteins and/or Antibodies

In various embodiments, the MTS may be attached to the IκB protein by means of a tag protein. Such proteins are well known in the field of fusion protein construction. See for example, Terpe, K. (Appl. Microbiol. Biotech. 60: 523-533, 2003). Table 1 shows typical examples of such tags. These can include but are not limited to poly-arginine, poly-histidine, calmodulin-binding peptide, cellulose-binding domain, protein disulfide isomerase I (DsbA), c-myc, glutathione S-transferase, a FLAG sequence, natural histidine tag (HAT), maltose-binding protein, transcript termination anti-termination factor (NusA), Staphylcoccal protein A, Staphylcoccal protein G, streptavidin binding peptide (SBP), S-RNAase (S) tag, Strep-tag, chitin-binding domain and thioredoxin or any combination thereof.

TABLE 1
Tag Proteins

| | | | SEQ |
|---|---|---|---|
| | | | ID |
| Tag | Residues | Sequence | NO. |
| Poly-Arg | 5-6 | Arg-Arg-Arg-Arg-Arg | NA |
| | (usually 5) | | |
| Poly-His | 2-10 | His-His-His-His-His-His | NA |
| | (usually 6) | | |
| FLAG | 8 | Asp-Tyr-Lys-Asp-Asp-Asp-Asp-Lys | 10 |
| Strep-tag II | 8 | Trp-Ser-His-Pro-Gln-Phe-Glu-Lys | 11 |
| c-myc | 11 | Glu-Gln-Lys-Leu-Ile-Ser-Glu-Glu- | 12 |
| | | Asp-Leu | |
| S- | 15 | Lys-Glu-Thr-Ala-Ala-Ala-Lys-Phe-Glu- | 13 |
| | | Arg-Gln-His-Met-Asp-Ser | |
| HAT- | 19 | Lys-Asp-His-Leu-Ile-His-Asn-Val-His- | 14 |
| | | Lys-Glu-Phe-His-Ala-His-Ala-His-Asn- | |
| | | Lys | |
| FLAG | 22 | Asp-Tyr-Lys-Asp-His-Asp-Gly-Asp- | 15 |
| | | Tyr-Lys-Asp-His-Asp-Ile-Asp-Tyr-Lys- | |
| | | Asp-Asp-Asp-Asp-Lys | |
| Calmodulin- | 26 | Lys-Arg-Arg-Trp-Lys-Lys-Asn-Phe-Ile- | 16 |
| binding | | Ala-Val-Ser-Ala-Ala-Asn-Arg-Phe-Lys- | |
| peptide | | Lys-Ile-Ser-Ser-Ser-Gly-Ala-Leu | |
| Cellulose- | 27-189 | Domains | NA |
| binding | | | |
| domains | | | |
| SBP | 38 | Met-Asp-Glu-Lys-Thr-Thr-Gly-Trp- | 17 |
| | | Arg-Gly-Gly-His-Val-Val-Glu-Gly-Leu- | |
| | | Ala-Gly-Glu-Leu-Glu-Gln-Leu-Arg- | |
| | | Ala-Arg-Leu-Glu-His-His-Pro-Gln-Gly- | |
| | | Gln-Arg-Glu-Pro | |
| Chitin- | 51 | Thr-Asn-Pro-Gly-Val-Ser-Ala-Trp-Gln- | 18 |
| binding | | Val-Asn-Thr-Ala-Tyr-Thr-Ala-Gly-Gln- | |
| domain | | Leu-Val-Thr-Tyr-Asn-Gly-Lys-Thr-Tyr- | |
| | | Lys-Cys-Leu-Gln-Pro-His-Thr-Ser-Leu- | |
| | | Ala-Gly-Trp-Glu-Pro-Ser-Asn-Val-Pro- | |
| | | Ala-Leu-Trp-Gln-Leu-Gln | |
| Glutathione | 211 | Protein | NA |
| S- | | | |
| transferase | | | |
| Maltose- | 396 | Protein | NA |
| binding | | | |
| protein | | | |

In alternative embodiments, the fusion protein or the fusion protein can be attached to an antibody which can enter the cell to facilitate the inhibition of immune responses or other mediated by NF-κB. The use of an MTS to deliver antibodies in this way has been shown to be an effective means of delivering antibodies into the cell without harming the cell. See for example, Zhao et al (J. Immunol. Methods 254:137-145, 2001).

Clinical Applications

The methods of the current invention can be used to prevent or treat an immune response that is the result of activation of the NF-κB/rel cascade. Immune reactions are often called hypersensitivity reactions and can be initiated either by the interaction of antigen with hormonal antibody or by cell-medicated immune mechanisms. Furthermore, immune reactions may be evoked by both exogeneous antigens and endogenous or intrinsic antigens. Administration of the fusion-protein-MST of the current invention can protect against the immune reactions by intrinsic antigens. Examples of such immune reactions mediated by intrinsic antigens include transfusion reactions, graft rejections and autoimmune diseases.

Thus, the fusion protein of the present invention may be used to treat various immune related diseases and conditions, such as T cell mediated diseases, including those characterized by infiltration of inflammatory cells into a tissue, stimulation of T-cell proliferation, inhibition of T-cell proliferation, increased or decreased vascular permeability or the inhibition thereof.

Thus, disorders or conditions to be treated or prevented with the fusion protein of the present invention, include, but are not limited to systemic lupus erythematosis, rheumatoid arthritis, juvenile chronic arthritis, osteoarthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In systemic lupus erythematosus, the central mediator of disease is the production of auto-reactive antibodies to self proteins/tissues and the subsequent generation of immune-mediated inflammation. Antibodies either directly or indirectly mediate tissue injury. Though T lymphocytes have not been shown to be directly involved in tissue damage, T lymphocytes are required for the development of auto-reactive antibodies. The genesis of the disease is thus T lymphocyte dependent. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing spondylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class 1 MHC); ocular inflammation, and absence of autoantibodies associated with other rheumatoid disease. The cell most implicated as key to induction of the disease is the CD8+T lymphocyte, a cell which targets antigen presented by class 1 MHC molecules. CD8+T cells may react against the class 1 MHC allele HLA-B27 as if it were a foreign peptide expressed by MHC class 1 molecules. It has been hypothesized that an epitope of HLA-B27 may mimic a bacterial or other microbial antigenic epitope and thus induce a CD8+T cell response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often up-regulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjögren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitis can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, or other mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type 1 diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including multiple sclerosis; idiopathic demyelinating polyneuropathy or Guillain-Barre syndrome; and chronic inflammatory demyelinating polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+ T lymphocytes are the predominant cell type at lesions. The mechanism of oligodendrocyte cell death and subsequent demyelination is not known but is likely T lymphocyte driven.

Inflammatory and fibrotic lung disease, including eosinophilic pneumonias; idiopathic pulmonary fibrosis, and hypersensitivity pneumonitis may involve a disregulated immune-inflammatory response. Inhibition of that response would be of therapeutic benefit.

Autoimmune or immune mediated skin disease including bullous skin diseases, erythema multiforme, and contact dermatitis are mediated by auto antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus Host Disease (GVHD) are T lymphocyte-dependent; inhibition of T lymphocyte function is ameliorative. Other diseases in which intervention of the immune and/or inflammatory response have benefit are infectious disease including but not limited to viral infection (including but not limited to AIDS, hepatitis A, B, C, D, E and herpes) bacterial infection, fungal infections, and protozoal and parasitic infections (molecules (or derivatives/agonists).

Additionally, inhibition of molecules with proinflammatory properties may have therapeutic benefit in reperfusion injury; stroke; myocardial infarction; atherosclerosis; acute lung injury; hemorrhagic shock; burn; sepsis/septic shock; acute tubular necrosis; endometriosis; degenerative joint disease and pancreatis.

Administration of the fusion-protein-MST of the current invention can also protect against the immune responses involved in hypersensitivity reactions. Hypersensitivity reactions can be divided in four basic classifications based upon the type of immunologic mechanism involved in the response. Type I responses involve the release of vasoactive and spasmogenic substances that act primarily on blood vessels and smooth muscle and pro-inflammatory cytokines that can act to recruit inflammatory cells. Type I hypersensitivity may be defined as a rapidly developing immunologic reaction occurring within minutes after the combination of an antigen with antibody bound to mast cells or basophils in individuals previously sensitized to the antigen.

Type II reactions involve humoral antibodies that participate directly in injuring cells through predisposing them to phagocytosis or lysis. Type II hypersensitivity is mediated by antibodies directed toward antigens present on the surface of cells or other tissue components. The antigenic determinants may be intrinsic to the cell membrane or they may take the form of an exogenous antigen such as a drug metabolite adsorbed on the cell surface. Non-limiting examples of Type II reactions include but are not limited to, transfusion reactions from blood donations, erythroblastosis fetalis in which there is an antigenic difference between the mother and the fetus and antibodies from the mother cross the placenta and cause destruction of fetal red blood cells, autoimmune hemolytic anemia, agranulocytosis, or thrombocytopenia and drug reactions where antibodies are produced that react with the drug which may be complexed to red cell antigens. Another clinical example of a Type II reaction includes myasthenia gravis in which antibodies are formed to the acetylcholine receptor at the motor end-plate in muscle.

Type III disorders involve immune reactions in which humoral antibodies bind antigens and activate the complement cascade. Type III hypersensitivity reactions are induced by antigen-antibody complexes that produce tissue damage as a result of their capacity to activate the compliment system. Generally two types of antigens cause immune complex mediated injury. These may be exogenous such as a foreign protein, bacterium or virus, or endogenous antigens in which antibodies are formed against antigenic components against an individuals own cells or tissue. Non-limiting examples of these types of disorders as discussed herein, include, but are not limited to, certain types of arthritis, hemolytic anemia, rheumatoid arthritis, pleuritis and systemic lupus erythematosus.

Type IV reactions involve tissue injury in which cell-medicated immune responses induce cellular and tissue injury. Type IV hypersensitivity reactions or cell mediated hypersensitivity is initiated by specifically sensitized T lymphocytes. These types of reactions include the classic delayed-type hypersensitivity reactions initiated by CD4 T cells and direct cell cytotoxicity mediated by CD8 T cells. Non-limiting examples of delayed type hypersensitivity discussed herein, include, but are not limited to, reactions to microbiological agents such as Mycobacterium tuberculosis, as well as other viruses, fungi and parasites. Included in these types of reactions are contact skin sensitivity to chemical agents (contact dermatitis) and graft rejections.

Administration of the fusion protein of the current invention can protect against immune reactions against self-antigens or so called autoimmunity. Examples of such autoimmune diseases as discussed herein, include, but are not limited to, systemic lupus erythematosus, rheumatoid arthritis, systemic sclerosis, insulin-dependent diabetes mellitus, myasthenia gravis, Graves disease, Sjögren syndrome, and inflammatory myopathies.

Another autoimmune-associated disease that can be treated with the current invention is amyloidosis. This disease is actually a family of diseases that all involve the deposition of amyloid, a pathologic proteinaceous substance between cells in various tissues and organs. With progressive accumulation, amyloid encroaches on and produces atrophy of adjacent cells. Amyloid deposition in the brain is one of the hallmark features of Alzheimer's disease. One form of amyloidosis is termed secondary or reactive amyloidosis and is thought to occur secondary to various chronic inflammatory conditions, including but not limited to rheumatoid arthritis, ankylosing spondylitis, and other connective tissue diseases. The chronic skin infections associated with the “skin-popping” or subcutaneous injection of heroin and other narcotics appear to be responsible for the high incidence of amyloidosis in heroin abusers.

Administration of the fusion proteins of the present invention can also be used to treat or protect against diseases that involve disregulation of cellular growth. The NF-κB family of transcription factors has also been demonstrated to play a role in regulating other cellular processes such as apoptotic cell death or in the development of cancer. These transcription factors can act as inducers or blockers of apoptosis in a stimulus- and cell type-dependent fashion. Studies have shown that these factors play a role in the embryonic responses to teratogens (Torchinsky et al., 2002).

Pharmaceutical Compositions

The fusion proteins of the present invention can be administered for the prevention or treatment of immune related diseases, in the form of pharmaceutical compositions. The fusion proteins of the present invention can be incorporated into pharmaceutical compositions suitable for administration. Such compositions typically comprise the fusion protein and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

Formulations of the fusion protein are prepared for storage by mixing the active protein having the desired degree of purity with optional pharmaceutically acceptable carriers, excipients or stabilizers (Remington's Pharmaceutical Sciences (2000), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS®, or polyethylene glycol (PEG).

The fusion protein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (2000).

One skilled in the art will also recognize that the formulations to be used for in vivo administration are formulated to be compatible with its intended route of administration. Examples of routes of administration include parenteral, e.g., intravenous, intradermal, subcutaneous, oral (e.g., inhalation or deep lung inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the fusion protein in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, capsules, powders, solutions or elixirs. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When administered in tablet form, the pharmaceutical composition of the invention may additionally contain a solid carrier such as a gelatin or an adjuvant. The tablet, capsule, and powder contain from about 5 to 95% fusion protein of the present invention, and preferably from about 25 to 90% fusion protein of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compositions can also be prepared in the form of suppositories (e.g., with conventional suppository bases such as cocoa butter and other glycerides) for vaginal or retention enemas for rectal delivery.

In one embodiment, the fusion proteins are prepared with carriers that will protect the fusion protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. The applicants also contemplate that other delivery systems can be utilized to administer the fusion protein of the present invention. One such delivery system is a an aerosol dry powder delivery system for deep lung administration. Such delivery systems are well known in the art, for example, as described in U.S. Pat. Nos. 6,254,854, 6,399,102, 6,436,443, 6,447,752, 6,447,753, 6,136,295, and 6,503,480, incorporated herein by reference. This delivery system involves a low density, porous particle structure with a geometric diameter of 5-30 μm and an aerodynamic diameter of 1-5 μm. These particles can be delivered using small, simple inhalers, can accommodate high drug doses and offer the potential for prolonged release. Thus, an efficient dry-powder delivery of the fusion protein to the deep lung can be accomplished allowing direct systemic or targeted delivery of the fusion protein which provides a rapid onset of action and the potential for prolonged release.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of fusion protein calculated to produce the desired therapeutic effect in association with the required pharmaceutical carrier. The specification for the dosage unit forms of the invention are dictated by and directly dependent on the unique characteristics of the fusion protein and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Generally, compounds or compositions which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

In practicing the method of treatment or use of the present invention, a therapeutically effective amount of protein of the present invention is administered to a host having a condition to be treated. The fusion protein of the present invention can be administered in accordance with the method of the invention either alone or in combination with other therapies such as treatments employing cytokines, lymphokines or other hematopoietic factors. When co-administered with one or more immunosupressive agents, anti-inflammatory drugs, cytokines, lymphokines or other hematopoietic factors, the fusion protein of the present invention can be administered either simultaneously with the immunosupressive agents, anti-inflammatory drugs, cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with immunosupressive agents, anti-inflammatory drugs, cytokine(s), lymphokine(s), other hematopoietic factor(s), thrombolytic or anti-thrombotic factors.

When a therapeutically effective amount of the fusion protein of the present invention is administered by intravenous, cutaneous or subcutaneous injection, the infusion protein of the present invention will be in the form of a pyrogen-free, parenterally acceptable aqueous solution. The preparation of such parenterally acceptable fusion protein solutions, having due regard to pH, isotonicity, stability, and the like, is within the skill in the art. A preferred pharmaceutical composition for intravenous, cutaneous, or subcutaneous injection should contain, in addition to the fusion protein of the present invention, an isotonic vehicle such as sodium chloride injection, ringer's injection, dextrose injection, dextrose and sodium chloride injection, Lactated Ringer's Injection, or other vehicle as known in the art. The pharmaceutical composition of the present invention may also contain stabilizers, preservatives, buffers, antioxidants, or other additives known to those of skill in the art.

The amount of fusion protein in the pharmaceutical compositions of the present invention will depend upon the nature and severity of the condition being treated, and on the nature of prior treatments which the patient has undergone. Ultimately, the attending physician will decide the amount of fusion protein of the present invention with which to treat each individual patient. Initially, the attending physician will administer low doses and observe the patient's response. Larger doses of the fusion protein of the present invention can be administered until the optimal therapeutic effect is obtained for the patient, and at that point the dosage is not increased further. It is contemplated that the various pharmaceutical compositions used to practice the method of the present invention should contain about 0.01 μg to about 100 mg of fusion protein per kg body weight. Preferably the composition will contain about 0.1 ng to about 10 mg. More preferably the composition will contain about 0.1 μg to about 1 mg).

The duration of intravenous therapy using the pharmaceutical composition of the present invention will vary, depending on the severity of the disease being treated and the condition and potential idiosyncratic response of each individual patient. It is contemplated that the duration of each application of the fusion protein of the present invention will be in the range of 12 to 24 hours of continuous intravenous administration. Ultimately the attending physician will decide on the appropriate duration of intravenous therapy using the pharmaceutical composition of the present invention.

Sustained-release preparations of the fusion protein may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The formulation herein may also contain more than one active compound as necessary for the particular indication being treated, preferably those with complementary activities, including but not limited to, an immunosuppressive agent anti-inflammatory drug or anti-histaminergic drug. Alternatively, or in addition, the composition may comprise a cytotoxic agent, cytokine or growth inhibitory agent. Such molecules are suitably present in combination in amounts that are effective for the purpose intended.

As indicated herein, supplementary active compounds can also be incorporated into the compositions. Thus, these other agents either enhance the activity of the fusion protein or compliment its activity or use in treatment. Thus, the pharmaceutical composition of the invention may also contain additional cytokines, lymphokines, anti-inflammatory agents, immunosuppressive drugs, antihistaminergic drugs or other hematopoietic factors such as, but not limited to, M-CSF, G-CSF, GM-CSF, Meg-GCSF, thrombopoietin, stem cell factor, erythropoietin, TNFα, IL-1β, IL-2 through IL-26, IFNα/b, IFNγ, as well as inhibitors of all of the above cytokines, particularly inhibitors of TNFα, IL-1β, IL-12 and IL-18.

Such additional factors and/or agents can be included in the pharmaceutical composition to produce a synergistic effect with the fusion protein of the invention, or to minimize side effects. Conversely, the fusion protein of the present invention can be included in formulations of the particular cytokine, lymphokine, other hematopoietic factor, thrombolytic or anti-thrombotic factor, immunosuppresive, or anti-inflammatory agent or antihistaminergic agent to minimize side effects of the particular drug.

Since endogenous histamine is released during the immediate stages following an immune challenge, antihistamine drugs are often administered to counteract the effects of histamine on tissues. Thus, antihistamine drugs would complement the effect of the fusion protein of the present invention to prevent or treat an immune response. Examples of antihistaminergic agents that can be included in the formulation or the fusion protein of the present invention or administered along with the fusion protein, include, but are not limited to, carbinoxamine, clemastine, diphenhydramine, dimenhydrinate, pyrilamine, tripelennamine, chlorpheniramine, brompheniramine, hydrazine, cyclizine, meclizine, promethazine, acrivastine, cetirizine, astemizole, levocabastine, loratadine and terfenadine or any pharmaceutically acceptable prodrug or derivative thereof.

It is further contemplated that the fusion protein of the present invention can be administered in conjunction with anti-inflammatory agents or procedures. Examples of anti-inflammatory agents that can be included in the formulation or the fusion protein of the present invention or administered along with the fusion protein, include, but are not limited to, aspirin, diflunisal, mesalamine, salicylsalicylic acid, sodium thiosalicylate, choline salicylate, magnesium salicylate, olsalazine, sulfasalazine, indomethacin, suldinac, etodolac, mefenamate, meclofenamate, flufenamate, tolfenamate, etofenamate, tolmetin, ketorolac, diclofenac, ibuprofen, naproxen, fenoprofen, ketoprofen, flurbiprofen, oxaprozin, piroxicam, meloxicam, nabumetone, apazone, nimesulide, zileuton, gold salts, colchicine, allopurinol, beclomethasone, budesonide, flunisolide, triamcinolone, prednisone, cromolyn, nedocromil, albuterol, bitolterol, pirbuterol, salmeterol, terbutaline, theophylline and other methylxanthines, metaproterenol, systemic glucocorticoids, antibiotics, antiparasitic agents, antiprotozoal agents, antimalarial agents, isoniazid, rifampin, ethambutol, antifungal agents, antiviral agents, alkylating agent, an antimetabolites, retinal, tretinoin, isotretinoin, etretinate, acitretin, arotinoid, β-carotene, calcipotriene, anthralin, psoralen, 5-methoxypsoralen, trioxsalen, coal tar, masoprocol, and any pharmaceutically acceptable prodrug or derivative thereof.

It is contemplated that the fusion protein disclosed herein could be combined therapeutically with an agent that induces immunosuppression in order to treat or prevent an immune response. The goal of immunosuppression is to control allograft rejection. Clinical immunosuppression involves non-specific suppression of both cell-mediated and humoral immunity. A number of methods have been proposed to control allograft prolongation. These methods have been developed from the understanding of the body's immune response to foreign antigen, and interaction of antigens, antibodies, macrophage, and lymphocytes. Currently, most approaches to immunosuppression are non-specific and focus on suppression of lymphocytic interaction and proliferation, and lymphocyte depletion. Examples of immunosuppressive agents that can be included in the formulation of the fusion protein of the present invention or administered along with the fusion protein, include, but are not limited to, cyclosporine, tacrolimus, azathioprine, mycophenolate, methotrexate, an immunoglobulin, a monoclonal antibody, an Rh(D) immune globulin, methoxsalen, thalidomide, radiation, or any pharmaceutically acceptable prodrug or derivative thereof.

Preparation and dosing schedules for any such agent disclosed herein that may be combined with the fusion protein of the present invention may be used according to manufacturers' instructions or as determined empirically by the skilled practitioner. The agent or agents to be combined with the fusion protein may precede or follow administration of the fusion protein or may be given simultaneously therewith.

For the treatment or reduction in the severity of immune related disease, the appropriate dosage of an a compound of the invention will depend on the type of disease to be treated, as defined above, the severity and course of the disease, whether the agent is administered for preventive or therapeutic purposes, previous therapy, the patient's clinical history and response to the compound, and the discretion of the attending physician. The compound is suitably administered to the patient at one time or over a series of treatments.

Without intent to limit the scope of the invention, exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as data are processed, sampled, converted, or the like according to the invention without regard for any particular theory or scheme of action.

### EXAMPLES

### Example 1

### Construction and Purification of IκBα-(ΔN)-Membrane Translocating Sequence Protein

An IκBα molecule was created by deletion of the amino terminal portion of the protein using the RT-PCR technique with RNA obtained from Jurkat T cells. To produce the fusion protein cDNA of IκBα-(ΔN) was cloned into an MTS vector that allows expression of the fusion protein tagged with GST on the N+ terminus of the protein and the MTS motif on the carboxyl terminus. Protein expression was induced by the addition of isopropyl β-D thiogalactoside (IPTG) to the culture.

cDNA from Jurkat T cells was used as a template to amplify specific cDNA of IκBα (Haskill et al., 1991). Each of the primers contained a BamHI site at the 5′-end. The sequence of the primers was:

(SEQ ID NO. 10)

5′-CCGGATCCCCATGAAAGACGAGGAGTACGAGCAGATGGTC
and

(SEQ ID NO. 11)

5′-CCGGATCCCTAACGTCAGACGCTGGCCTCCAAACACACA.

These primers encode a cDNA for NH₂ terminal truncated form (amino acid 37-317) of IκBα. The PCR product was originally cloned in pGEMT-easy (Promega, Wis.) and then sub-cloned into the MTS expression vector to produce a cell permeable fusion protein called, glutathione S-transferase (GST)-IκBα-(ΔN)-MTS (Rojas et al., 1998). As a control, a non-permeable GST-IκBα-(ΔN) without the MTS sequence was produced in the expression vector pGEX-3×. Translation and reading frames were confirmed by automated sequence.

Recombinant cell permeable IκBα-(ΔN)-MTS and non-permeable proteins were purified by a single step chromatography using a glutathione agarose bead column to isolate the protein from a bacterial lysate as follows. BL-21(DE3)RP bacteria strain (Stratagene, Calif.) transfected with the plasmids containing the IκBα protein were grown in 100 ml of Luria-Bertani (LB) broth containing 100 μg/ml of ampicillin. This overnight culture was inoculated in 900 ml of LB-Amp and growth at 37° C. until A₆₆₀ of O.D.=1.0. GST-fusion protein expression was induced by the addition of IPTG to final concentration of 0.5 mM (Sigma, Mo.), and incubation was continued for 3 h. GST fusion proteins were purified from bacterial cell lysates by glutathione agarose (Sigma, Mo.) chromatography and eluted with 5 mM reduced glutathione (Sigma, Mo.). Purified proteins were concentrated and washed with sterile PBS by ultrafiltration (Amicon, Calif.) and stored at 4° C. for immediate use or at −70° C. for prolonged storage. Protein concentration was determined in a spectrophotometer at 280 nm and purity corroborated by SDS-PAGE.

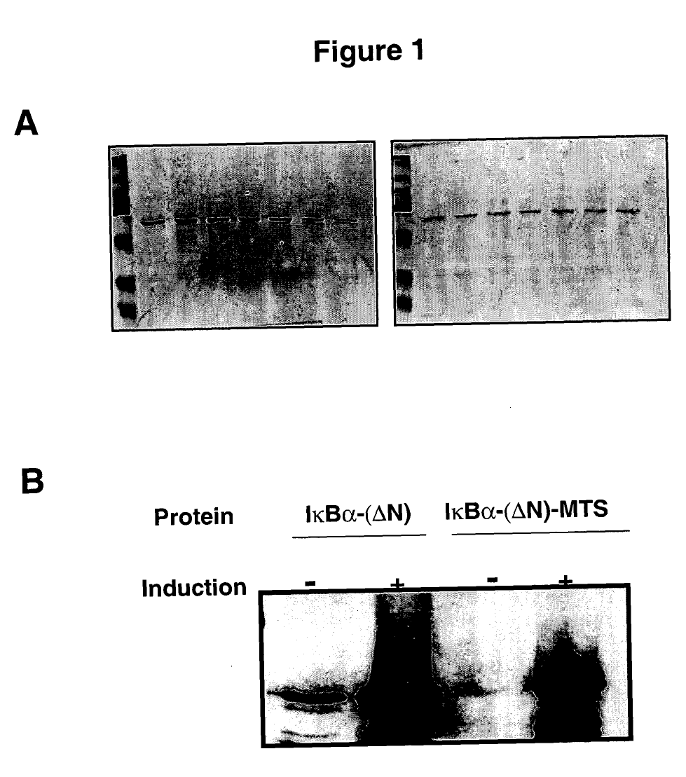

In the initial purification 7 fractions of 1 ml each were collected and tested by SDS-PAGE to determine concentration and purity. (Millipore, Calif.) (FIG. 1A). Fractions were combined and concentrated in an Amicon system. Two to 4 mg of protein were obtained per 1000 ml of bacterial culture. The yield of the permeable protein was 40 to 50% lower than the non-permeable protein.

To demonstrate that recombinant proteins maintain their antigenicity, the proteins were subjected to western blot analysis using a specific antibody against the carboxi-terminal portion of the IκBα molecule (FIG. 1B). After delivering the IκBα-(ΔN)-MTS protein, Jurkat T cells were activated with the combination of phorbol 12 myristate 13 acetate (PMA) and ionomycin. Activated cells were harvested and cytoplasmic extracts were obtained. Protein concentration was determined with a colorimetric assay (Bio-Rad, CA). Equal amounts of proteins were mixed with a pre-warmed sample buffer. Proteins were separated by SDS-PAGE, transferred to a nitrocellulose membrane membrane, and probed with anti-p50 and p65 antibody (Santa Cruz Biotech., CA). Antigen-antibody complexes were detected with an anti-rabbit serum coupled to horseradish peroxidase (Santa Cruz Biotech., CA) and developed by using an enhanced chemiluminescence system (Pierce, IL). Immunoblotting experiments detected the recombinant cell permeable fusion proteins and non-permeable proteins, indicating the preservation of antigenic epitopes in the cell permeable recombinant protein.

### Example 2

### Delivery of IκBα-(ΔN)-MTS Into Living Cells

To confirm the uptake of the recombinant protein, mammalian NIH3T3 fibroblast cells were used. Subconfluent NIH3T3 cells were incubated for 1 h with different concentrations of the wild type and cell permeable IκBα-(ΔN). Cells were fixed and protein was detected using an anti-GST antibody and a secondary antibody coupled to Texas red fluorochrome. Briefly, NIH3T3 cells were cultured in 4 well slides (Nunc, Calif.) and grown for 3 days at 37° C. Sub-confluent cells were washed with media without sera and incubated with the different proteins at a concentration of 90 μg/ml for 1 h at 37° C. The cells were washed with cold PBS and fixed with 3.7% paraformaldehyde in PBS at 37° C. for 15 min. Cells were washed again 3 times with PBS, and treated with 0.25% Triton X-100 in PBS for 10 min. Then, cells were incubated with blocking solution (PBS+1% normal goat serum (Sigma, Mo.)+1% BSA (Sigma, Mo.)) for 30 min at 37° C. and 5% CO₂. After blocking, cells were incubated with anti-GST (Santa Cruz Biotech. CA) in PBS+1% BSA for 2 h. Cells were washed 3 times with PBS and blocked for 30 min with blocking solution. Intracellular protein-antibody complexes were detected by incubating for 1 h with goat anti-rabbit IgG label with Texas Red (Molecular Probes, OR). Nuclei were stained with DAPI 1:10000 (Molecular Probes, OR) in 2% SSC for 1 min at room temperature. Coverslips were mounted in ProLong Antifade (Molecular Probes, OR) and analyzed in a fluorescence microscope (Olympus, NY).

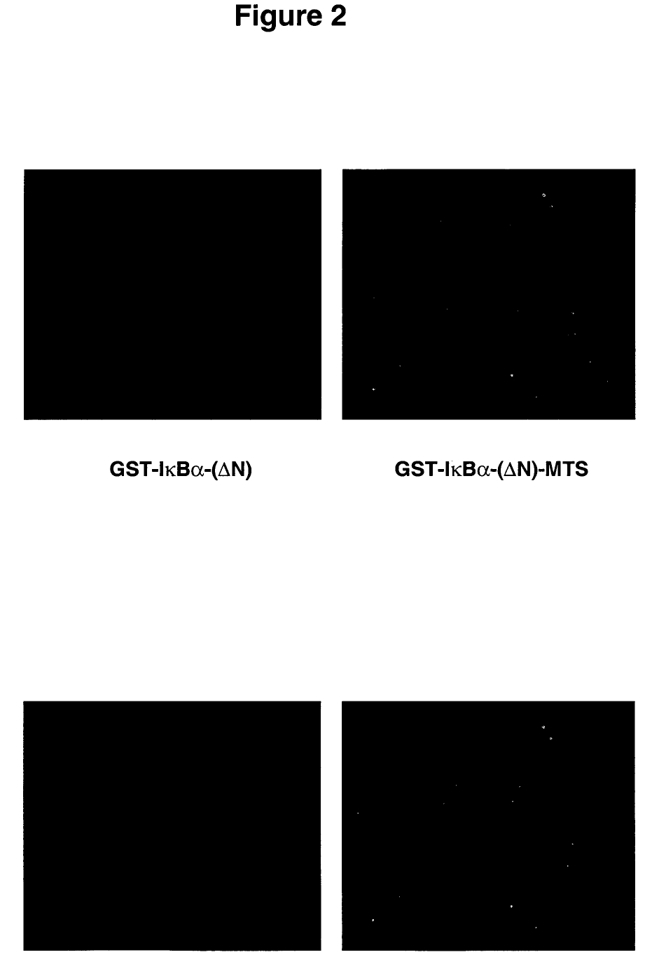

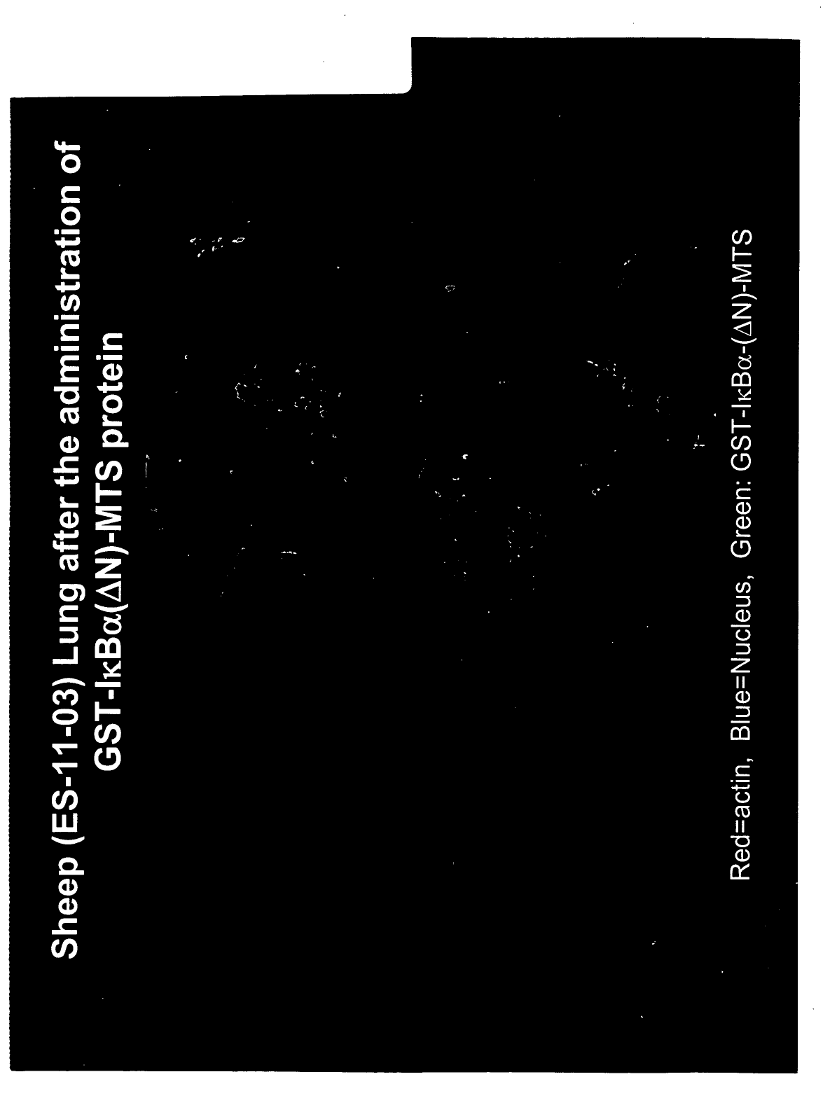

Cells treated with the permeable protein show an intra-cytoplasmic localization of the GST-IκBα-(ΔN)-MTS protein (FIG. 2). No signal was detected in cells incubated with the non-permeable protein. The amount of protein imported depended on the incubation time and the extracellular concentration of the permeable protein. Similarly, successful delivery of GST-IκBα-(ΔN)-MTS in an aerosol form to lung cells in a sheep has also been observed (FIG. 7).

### Example 3

### Inhibition of NF-κB Activity In Living Cells

The IκBα-(ΔN) molecule lacks the sequences required for signal-dependent degradation and it has been show in in vivo systems to be a constitutive repressor of multiple NF-κB/Rel proteins (Brockman et al., 1995; Boothby et al., 1997; Mora et al., 1999; Mora et al., 2001a; Mora et al., 2001b). In the absence of phosphorylation sites, IκBα protein is resistant to degradation but maintains the ability to interact with latent NF-κB/Rel complexes in the cytoplasm inducing permanent retention of NF-κB dimers in the cytoplasm.

To determine whether IκBα-(ΔN)-MTS inhibits endogenous NF-κB/Rel signaling pathway in vivo, mobility shift analyses in primary thymocytes were performed. Cell preparations were incubated for 1 h with the wild type and permeable recombinant proteins, followed by treatment with PMA/ionomycin. Chemical activation by PMA and ionomycin has been shown to mimic activation through the TCR-associated nuclear translocation of NF-κB proteins. Briefly, primary thymocytes from C57BL/6 mice, were incubated with the IκBα-(ΔN)-MTS protein, and activated with PMA/ionomycin to induce nuclear translocation of NF-κB. Nuclear fractions were prepared by high salt extraction in the presence of protease inhibitors. Gel mobility shift assays of NF-κB/Rel proteins were performed as previously have been described using a double-stranded ³²P_labeled oligonucleotide modified from κB enhancer sequences in the IL-2Rα promoter (KB-pd; upper strand, 5′-CAACGGCAGGGGAATTCCCCT-CTCCTT) (Ballard et al., 1990; Boothby et al., 1997). DNA binding reaction mixtures (20 μl) containing 4 μg of nuclear extract, 2 μg of double-stranded poly(dI-dC), 10 μg of BSA buffered in 20 mM HEPES (pH 7.9), 5% glycerol, 1 mM EDTA, 1% Nonidet P-40, and 5 mM DTT were then resolved on native 5% polyacrylamide gels and visualized by autoradiography.

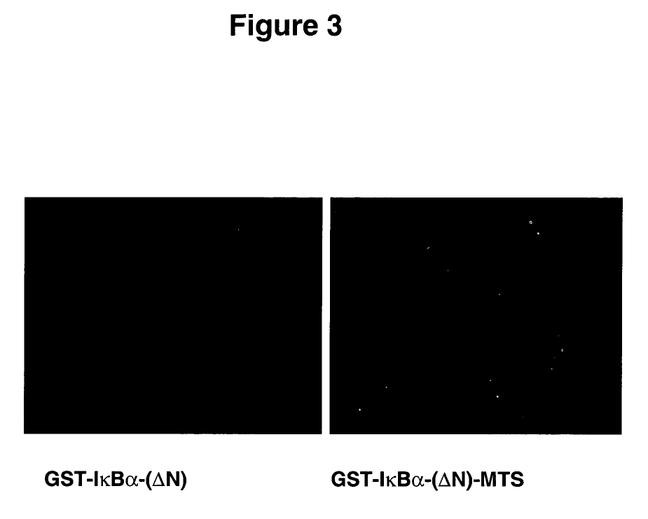

As shown in FIG. 3, nuclear translocation of NF-κB complexes was detected only in untreated cells or in cells pre-treated with the non-permeable IκBα-(ΔN) protein. Cells treated with IκBα-(ΔN)-MTS, showed a dose dependent, significant reduction of the DNA/protein complex, suggesting sequestration of the complex in the cytoplasm. Similar data were obtained using Jurkat T cells (data not shown).

The cytoplasmic retention of NF-κB proteins by IκBα-(ΔN)-MTS was also demonstrated. Jurkat T cells were pre-treated with permeable and non-permeable IκBα-(ΔN) and activated with PMA plus ionomycin. Cytoplasmic extracts were prepared and subjected to western blot analyses using antibodies specific against p65 and p50 NF-κB proteins. Cells untreated and treated with non-permeable protein showed a decrease in cytoplasmic concentration of p65 and p50 proteins. In contrast, cells treated with the permeable inhibitor showed, constant amount of NF-κB/Rel proteins in the cytoplasm. These results suggest that the delivered protein is inhibiting the translocation of the NF-κB complex from cytoplasm to the nucleus.

### Example 4

### Inhibition of NF-κB Cascade By The IκBα-(ΔN)-MTS Inhibits Growth Signal in Activated Primary T Cells

Inhibition of NF-κB pathways by the transgenic expression of the inhibitor IκBα-(ΔN) has been reported to decrease synthesis of DNA (Boothby et al., 1997; Mora et al., 2001a). To determine if IκBα-(ΔN)-MTS imported in primary T cells has similar effects, CD4⁺ T cells derived from DO. 11.10 TCR transgenic mice that respond specifically to the OVA₃₂₃₋₃₃₉ peptide were used.

Briefly, DO. 11 TCR transgenic mice were used as a source of T cells specific for a known antigen (OVA₃₂₃₋₃₃₉); (Murphy et al., 1990). Antigen presenting cells were obtained from adherent spleen cells isolated from wild type BALB/c mice. DO.11 CD4⁺ T cells were obtained by negative selection using anti-CD8+ and anti-class II magnetic coated beads (Miltenyl). Purified CD4+cells were incubated at 37° C. with 250 μg/ml of the permeable recombinant protein. After one hour of treatment, cells were washed twice with sterile PBS and 1×10⁵ cells/well, plated simultaneously with 2×10⁵ APC cells. OVA₃₂₃₋₃₃₉ peptide was used at concentrations of 0.1, 0.5 and 1 μg/ml, and cultured 48 h at 37° C. and 5% CO₂. 3[H]+thymidine (0.1 μg/well) was added and cells cultured for an additional 12 h. Thymidine incorporation was measured in a beta counter and data was presented as counts per minute.

After 48 hours of stimulation control cells not treated with recombinant protein showed a significant increase in the amount of DNA synthesis determined by thymidine incorporation. Comparable responses were obtained on cells pre-treated with the non-permeable inhibitor IκBα-(ΔN). Only cells treated with the permeable IκBα-(ΔN)-MTS showed no increase in thymidine incorporation suggesting an inhibition in the delivery of the NF-κB-mediated signal to induce T cell expansion. Thus, the delivery of IκBα-(ΔN) permeable proteins into primary T cells altered the normal response of T lymphocytes to antigen stimulation.

### Example 5

### In Vivo Inhibition and NF-κB Gene Expression

To address the issue of in vivo activity of the recombinant proteins, a transgenic mouse (HLL) that expresses in every tissue a luciferase reporter gene driven by an NF-κB dependent promoter was used (Blackwell et al., 2000). Data in vivo in this model, show that luciferase production depends on NF-κB-activated gene transcription, making the HLL mouse a unique in vivo reporter-based assay system in which to analyze NF-κB. This mouse expresses a proximal 5′ human immunodeficiency virus (HIV-1) long terminal repeat (LTR) driving the expression of luciferase in all tissues (Blackwell et al., 2000). Mice were housed and cared for in accordance with the Guide for the Care and Use of Laboratory Animals [DHEW (DHHS) publication no. (NIH) 85-23 revised 1996, Office of Science and Health Reports. NIH].

Mice were anesthetized and the abdomen shaved. Two 1.5 cm long superficial incisions were made in the skin of the upper and lower portions of the abdomen. The incisions were closed with 2 stitches using a 4-0 suture. Fusion proteins were administrated I.P 12 and 2 hours before imaging (250 μg/dose in 0.5 ml of PBS). Luciferin (50 μg/mouse in 100 μl of PBS) was administered intravenously, and mice were imaged immediately with an intensified charge-coupled device (ICCD) camera (model C2400-32; Hamamatsu, Bridgewater, N.J.). This system consists of an image intensifier coupled to an 8-bit charge-coupled device camera, allowing for 256 intensity levels for each pixel. For the duration of photon counting, mice were placed inside a light tight box that also houses the camera. Light emission from the mouse was detected as photon counts using the ICCD camera and customized image processing hardware and software (Hamamatsu). Quantitative analysis was accomplished by defining a standard area (region of interest) in the 8-bit intensity image corresponding to the region of the abdomen overlying the incision and determination of total integrated photon intensity over the region of interest. For presentation, a 4-bit (16 intensity levels) digital false-color photon emission image was generated for each captured image according to the same false-color scale. To visualize the dimmer parts of the image, the brighter pixels in the images are displayed as white (thus appearing saturated); however, detected light emission for each image was well below the saturation limit for the camera.

In animals inoculated by an injection of the recombinant protein 24 hours after surgery, there was a reduction in the amount of light emitted by the animals that received the permeable form of the inhibitor. Because luciferase expression is driven by NF-κB activity, these results suggest that IκBα-(ΔN)-MTS was able, after a systemic inoculation, to reduce local NF-κB activation induced by the inflammatory process during skin injury.

The above specification, examples and data provide a complete description of the manufacture and use of the composition of the invention. Since many embodiments of the invention can be made without departing from the spirit and scope of the invention, the invention resides in the claims hereinafter appended.

### LIST OF REFERENCES

Aronica, M. A., Mora, A. L., Mitchell, D. B., Finn, P. W., Johnson, J. E., Sheller, J. R. and Boothby, M. R. (1999) Preferential role for NF-κB/Rel signaling in the type 1 but not type 2 T cell-dependent immune response in vivo. J Immunol 163, 5116-24.
Baldwin, A. S., Jr. (1996) The NF-κB and IκB proteins: new discoveries and insights. Annu Rev Immunol 14, 649-83.
Baldwin, A. S., Jr. (2001) Series introduction: the transcription factor NF-κB and human disease. J Clin Invest 107, 3-6.
Ballard, D. W., Walker, W. H., Doerre, S., Sista, P., Molitor, J. A., Dixon, E. P., Peffer, N. J., Hannink, M. and Greene, W. C. (1990) The v-rel oncogene encodes a κB enhancer binding protein that inhibits NF-κB function. Cell 63, 803-14.
Blackwell, T. S., Yull, F. E., Chen, C. L., Venkatakrishnan, A., Blackwell, T. R., Hicks, D. J., Lancaster, L. H., Christman, J. W. and Kerr, L. D. (2000) Multiorgan nuclear factor κB activation in a transgenic mouse model of systemic inflammation. Am J Respir Crit Care Med 162, 1095-101.
Boothby, M. R., Mora, A. L., Scherer, D. C., Brockman, J. A. and Ballard, D. W. (1997) Perturbation of the T lymphocyte lineage in transgenic mice expressing a constitutive repressor of nuclear factor (NF)-κB. J Exp Med 185, 1897-907.
Brockman, J. A., Scherer, D. C., McKinsey, T. A., Hall, S. M., Qi, X., Lee, W. Y. and Ballard, D. W. (1995) Coupling of a signal response domain in I κ B alpha to multiple pathways for NF-κB activation. Mol Cell Biol 15, 2809-18.
Deutscher, M. P. (1982) Rethinking your purification procedure. Methods Enzymology 182, 779-800.
Du, C., Yao, S., Rojas, M. and Lin, Y. Z. (1998) Conformational and topological requirements of cell-permeable peptide function. J Pept Res 51, 235-43.
Epinat, J. C. and Gilmore, T. D. (1999) Diverse agents act at multiple levels to inhibit the Rel/NF-κB signal transduction pathway. Oncogene 18, 6896-909.
Fernandez, T. and Bayley, H. (1998) Ferrying proteins to the other side. Nat Biotechnol 16, 418-20.
Finco, T. S. and Baldwin, A. S. (1995) Mechanistic aspects of NF-κB regulation: the emerging role of phosphorylation and proteolysis. Immunity 3, 263-72. Ghosh, S., May, M. J. and Kopp, E. B. (1998) NF-κB and Rel proteins: evolutionarily conserved mediators of immune responses. Annu Rev Immunol 16, 225-60.
Haskill, S., Beg, A. A., Tompkins, S. M., Morris, J. S., Yurochko, A. D., Sampson-Johannes, A., Mondal, K., Ralph, P. and Baldwin, A. S., Jr. (1991) Characterization of an immediate-early gene induced in adherent monocytes that encodes I κ B-like activity. Cell 65, 1281-9.
Jo, D., Nashabi, A., Doxsee, C., Lin, Q., Unutmaz, D., Chen, J. and Ruley, H. E. (2001) Epigenetic regulation of gene structure and function with a cell-permeable Cre recombinase. Nat Biotechnol 19, 929-33.
Kaiser, J. (2003) Gene therapy. Seeking the cause of induced leukemias in X-SCID trial. Science 299, 495.
Karin, M. (1999) How NF-κB is activated: the role of the IκB kinase (IKK) complex. Oncogene 18, 6867-74.
Li, Q. and Verma, I. M. (2002) NF-κB regulation in the immune system. Nat Rev Immunol 2, 725-34.
Lin, Y. Z., Yao, S. Y. and Hawiger, J. (1996) Role of the nuclear localization sequence in fibroblast growth factor-1-stimulated mitogenic pathways. J Biol Chem 271, 5305-8.
Lin, Y. Z., Yao, S. Y., Veach, R. A., Torgerson, T. R. and Hawiger, J. (1995) Inhibition of nuclear translocation of transcription factor NF-κB by a synthetic peptide containing a cell membrane-permeable motif and nuclear localization sequence. J Biol Chem 270, 14255-8.
Liu, K. Y., Timmons, S., Lin, Y. Z. and Hawiger, J. (1996) Identification of a functionally important sequence in the cytoplasmic tail of integrin beta 3 by using cell-permeable peptide analogs. Proc Natl Acad Sci USA 93, 11819-24.
Marshall, E. (2002a) Clinical research. Gene therapy a suspect in leukemia-like disease. Science 298, 34-5.
Marshall, E. (2002b) Gene therapy. What to do when clear success comes with an unclear risk? Science 298, 510-1.
Marshall, E. (2003) Gene therapy. Second child in French trial is found to have leukemia. Science 299, 320.
Mora, A., Youn, J., Keegan, A. and Boothby, M. (2001 a) NF-κB/Rel participation in the lymphokine-dependent proliferation of T lymphoid cells. J Immunol 166, 2218-27.
Mora, A. L., Chen, D., Boothby, M. and Rubin, D. H. (1999) Lineage-specific differences among CD8+T cells in their dependence of NF-κB/Rel signaling. Eur J Immunol 29, 2968-80.
Mora, A. L., Stanley, S., Armistead, W., Chan, A. C. and Boothby, M. (200 lb) Inefficient ZAP-70 phosphorylation and decreased thymic selection in vivo result from inhibition of NF-κB/Rel. J Immunol 167, 5628-35.
Murphy, K. M., Heimberger, A. B. and Loh, D. Y. (1990) Induction by antigen of intrathymic apoptosis of CD4+CD8+TCR10 thymocytes in vivo. Science 250, 1720-3.
Pahl, H. L. (1999) Activators and target genes of Rel/NF-κB transcription factors. Oncogene 18, 6853-66.
Remington's Pharmaceutical Sciences, Gennaro AR ed. 20th edition, 2000: Williams & Wilkins Pa., USA.
Rojas, M., Donahue, J. P., Tan, Z. and Lin, Y. Z. (1998) Genetic engineering of proteins with cell membrane permeability. Nat Biotechnol 16, 370-5.
Rojas, M., Yao, S., Donahue, J. P. and Lin, Y. Z. (1997) An alternative to phosphotyrosine-containing motifs for binding to an SH2 domain. Biochem Biophys Res Commun 234, 675-80.
Rojas, M., Yao, S. and Lin, Y. Z. (1996) Controlling epidermal growth factor (EGF)-stimulated Ras activation in intact cells by a cell-permeable peptide mimicking phosphorylated EGF receptor. J Biol Chem 271, 27456-61.
Scopes, R. K., Stoter, A (1982) Purification of all glycolytic enzymes from one muscle extract. Methods Enzymology 90 Pt.E:479-90.
Seetharaman, R., Mora, A. L., Nabozny, G., Boothby, M. and Chen, J. (1999) Essential role of T cell NF-κB activation in collagen-induced arthritis. J Immunol 163, 1577-83.
Tam, J. P., Lu, Ya, Lin C. F., Shao, J., Peptide synthesis using unprotected peptides through orthogonal coupling methods (1995) Proc. Natl. Acad. Sci USA, 92-12485-12489.
Terpe, K. (2003) Overview of tag fusion proteins: from molecular and biochemical fundamentals to commercial systems. Appl. Microbiol. Biotechnol. 60: 523-533.
Torchinsky et al, NF-κB DNA-binding activity in embryos responding to a teratogen, cyclophosphamide, BMC Developmental Biology 2002, 2:2.
Turkson, J., Ryan, D., Kim, J. S., Zhang, Y., Chen, Z., Haura, E., Laudano, A., Sebti, S., Hamilton, A. D. and Jove, R. (2001) Phosphotyrosyl peptides block Stat3-mediated DNA binding activity, gene regulation, and cell transformation. J Biol Chem 276, 45443-55.
Verma, I. M., Stevenson, J. K., Schwarz, E. M., Van Antwerp, D. and Miyamoto, S. (1995) Rel/NF-κB/I κ B family: intimate tales of association and dissociation. Genes Dev 9, 2723-35.
Wang, S., Bartido, S., Yang, G., Qin, J., Moroi, Y., Panageas, K. S., Lewis, J. J. and Houghton, A. N. (1999) A role for a melanosome transport signal in accessing the MHC class II presentation pathway and in eliciting CD4+ T cell responses. J Immunol 163, 5820-6.
Wu, Y., Temple, J., Zhang, R., Dzhura, I., Zhang, W., Trimble, R., Roden, D. M., Passier, R., Olson, E. N., Colbran, R. J. and Anderson, M. E. (2002) Calmodulin kinase II and arrhythmias in a mouse model of cardiac hypertrophy. Circulation 106, 1288-93.
Yamamoto, Y. and Gaynor, R. B. (2001) Therapeutic potential of inhibition of the NF-κB pathway in the treatment of inflammation and cancer. J Clin Invest 107, 135-42.
Zhao, Y., Lou, D., Burkett, J. and Kohler, H. (2001) Chemical engineering of cell penetrating antibodies. J Immunol Methods 254, 137-45.

1. An isolated fusion protein comprising:
(a) a membrane-translocating sequence comprising at least eight consecutive residues of SEQ ID NO: 1 (Ala-Ala-Val-Leu-Leu-Pro-Val-Leu-Leu-Ala-Ala-Pro), and
(b) an IκB protein.

2. The isolated fusion protein of claim 1, wherein the membrane-translocating sequence comprises at least nine consecutive residues of SEQ ID NO: 1.

3. The isolated fusion protein of claim 1, wherein the membrane-translocating sequence comprises at least ten consecutive residues of SEQ ID NO: 1.

4. The isolated fusion protein of claim 1, wherein the membrane-translocating sequence comprises at least eleven consecutive residues of SEQ ID NO: 1.

5. The isolated fusion protein of claim 1, wherein the membrane-translocating sequence comprises at least twelve consecutive residues of SEQ ID NO: 1.

6. The isolated fusion protein of claim 1, wherein the IκB protein comprises an IκBα protein.

7. The isolated fusion protein of claim 1, wherein the IκB protein comprises an IκBβ protein.

8. The isolated fusion protein of claim 1, wherein the IκB protein comprises an IκBεε protein.

9. The isolated fusion protein of claim 1, wherein the IκB protein comprises a complex formed by two or more IκB proteins.

10. The isolated fusion protein of claim 1 further comprising a tag amino acid sequence or protein.

11. The isolated fusion protein of claim 10, wherein the tag amino acid sequence is selected from the group consisting of poly-arginine, poly-histidine, calmodulin-binding peptide (SEQ ID NO. 16), cellulose-binding domain, protein disulfide isomerase I (DsbA), c-myc (SEQ ID. NO. 12), glutathione S-transferase, a FLAG sequence (SEQ ID NO. 10), natural histidine tag (HAT; SEQ ID NO. 14), maltose-binding protein, transcript termination anti-termination factor (NusA), Staphylcoccal protein A, Staphylcoccal protein G, S—RNAase tag (SEQ ID NO. 13), streptavidin binding peptide (SEQ ID NO. 17), Strep-tag (SEQ ID. NO. 11), chitin-binding domain (SEQ ID NO. 18) and thioredoxin or any combination thereof.

12. The isolated fusion protein of claim 1 further comprising an antibody.

13. A pharmaceutical composition comprising:
(a) an isolated fusion protein having:
(1) a membrane-translocating sequence comprising at least eight consecutive residues of SEQ ID NO: 1 (Ala-Ala-Val-Leu-Leu-Pro-Val-Leu-Leu-Ala-Ala-Pro), and
(2) an IκB protein; and
(b) a pharmaceutically acceptable carrier.

14. The pharmaceutical composition of claim 13, wherein the isolated fusion protein further comprises a tag amino acid sequence or protein.

15. The pharmaceutical composition of claim 14, wherein the tag amino acid sequence is selected from the group consisting of poly-arginine, poly-histidine, calmodulin-binding peptide (SEQ ID NO. 16), cellulose-binding domain, protein disulfide isomerase I (DsbA), c-myc (SEQ ID. NO. 12), glutathione S-transferase, a FLAG sequence (SEQ ID NO. 10), natural histidine tag (HAT; SEQ ID NO. 14), maltose-binding protein, transcript termination anti-termination factor (NusA), Staphylcoccal protein A, Staphylcoccal protein G, S-RNAase tag (SEQ ID NO. 13), streptavidin binding peptide (SEQ ID NO. 17), Strep-tag (SEQ ID. NO. 11), chitin-binding domain (SEQ ID NO. 18) and thioredoxin or any combination thereof.

16. The pharmaceutical composition of claim 13, wherein the isolated fusion protein further comprises an antibody.

17. A method for preventing an immune response in a host comprising administration of an isolated fusion protein, wherein the isolated fusion protein comprises:
(a) a membrane-translocating sequence of about 8 to about 50 residues comprising at least eight consecutive residues of SEQ ID NO: 1 (Ala-Ala-Val-Leu-Leu-Pro-Val-Leu-Leu-Ala-Ala-Pro), and
(b) an IκB protein.

18. The method of claim 17, wherein the membrane-translocating sequence comprises at least nine consecutive residues of SEQ ID NO: 1.

19. The method of claim 17, wherein the membrane-translocating sequence comprises at least ten consecutive residues of SEQ ID NO: 1.

20. The method of claim 17, wherein the membrane-translocating sequence comprises at least eleven consecutive residues of SEQ ID NO: 1.

21. The method of claim 17 wherein the membrane-translocating sequence comprises at least twelve consecutive residues of SEQ ID NO: 1.

22. The method of claim 17, wherein the IκB protein comprises an IκBα protein.

23. The method of claim 17, wherein the IκB protein comprises an IκBβ protein.

24. The method of claim 17, wherein the IκB protein comprises an IκBεε protein.

25. The method of claim 17, wherein the IκB protein comprises a complex formed by two or more IκB proteins.

26. The method of claim 17, wherein the isolated fusion protein further comprises a tag amino acid sequence or protein.

27. The method of claim 26, wherein the tag amino acid sequence is selected from the group consisting of poly-arginine, poly-histidine, calmodulin-binding peptide (SEQ ID NO. 16), cellulose-binding domain, protein disulfide isomerase I (DsbA), c-myc (SEQ ID. NO. 12), glutathione S-transferase, a FLAG sequence (SEQ ID NO. 10), natural histidine tag (HAT; SEQ ID NO. 14), maltose-binding protein, transcript termination anti-termination factor (NusA), Staphylcoccal protein A, Staphylcoccal protein G, S-RNAase tag (SEQ ID NO. 13), streptavidin binding peptide (SEQ ID NO. 17), Strep-tag (SEQ ID. NO. 11), chitin-binding domain (SEQ ID NO. 18) and thioredoxin or any combination thereof.

28. The method of claim 17, wherein the isolated fusion protein further comprises an antibody.

29. The method of claim 17, wherein the immune response is associated with at least one of an allergy, asthma, contact dermatitis, delayed-type hypersensitivity, a wound-healing, allergic rhinitis, food hypersensitivity, ectopic dermatitis, imflammatory bowel disease, an immunologic disease of the lung, eosinophilic pneumonias, idiopathic pulmonary fibrosis, hypersensitivity pneumonitis, an autoimmune or immune-mediated skin disease, a bullous skin disease, erythemia multiforme, psoriasis, gluten-sensitive enteropathy, Whipple's disease, systemic lupus erythematisis, rheumatoid arthritis, osteoarthritis, juvenile chronic arthritis, ankylosing spondlylitis, systemic sclerosis, an idiopathic inflammatory myopathy, Sjögren's disease, pleuritis, sarcoidosis, amyloidisis, autoimmune hemolytic anemia, autoimmune thrombocytopenia, thyroiditis, diabetes mellitus, immune-mediated renal disease, myasthenia gravis, a demylenating disease of the central or peripheral nervous system, idiopathic demylenating polyneuropathy, Guillain-Barre syndrome, a chonic inflammatory demyelinating polyneuropathy, a hepotobiliary disease, an infectious or autoimmune chronic active hepatitis, primary biliary cirrhosis, granulomatous hepatitis, sclerlosing cholangitis, Graves disease, a transplantation-associated disease, a graft rejection, and graft-versus-host disease.

30. The method of claim 17, wherein the immune response is caused by exposure to Mycoplasma tuberculosis.

31. The method of claim 17, wherein the isolated fusion protein is administered prophylactically.

32. The method of claim 17, wherein the isolated fusion protein is administered therapeutically.

33. The method of claim 17, wherein the isolated fusion protein is administered by one or more of the following routes selected from the group consisting of intravenous, intradermal, subcutaneous, oral, inhalation, deep lung inhalation, transdermal, transmucosal, vaginal and rectal administration.

34. The method of claim 17, wherein the isolated fusion protein is administered as a liposome.

35. The method of claim 17, wherein the isolated fusion protein is administered as an aerosol.

36. The method of claim 17, wherein the infusion protein is administered in combination with a compound used to treat or prevent any immune-related disorder.

37. The method of claim 36, wherein the compound is an anti-inflammatory agent.

38. The method of claim 37, wherein the anti-inflammatory agent is selected from the group consisting of aspirin, diflunisal, mesalamine, salicylsalicylic acid, sodium thiosalicylate, choline salicylate, magnesium salicylate, olsalazine, sulfasalazine, indomethacin, suldinac, etodolac, mefenamate, meclofenamate, flufenamate, tolfenamate, etofenamate, tolmetin, ketorolac, diclofenac, ibuprofen, naproxen, fenoprofen, ketoprofen, flurbiprofen, oxaprozin, piroxicam, meloxicam, nabumetone, apazone, nimesulide, zileuton, gold salts, colchicine, allopurinol, beclomethasone, budesonide, flunisolide, triamcinolone, prednisone, cromolyn, nedocromil, albuterol, bitolterol, pirbuterol, salmeterol, terbutaline, theophylline and other methylxanthines, metaproterenol, systemic glucocorticoids, antibiotics, antiparasitic agents, antiprotozoal agents, antimalarial agents, isoniazid, rifampin, ethambutol, antifungal agents, antiviral agents, alkylating agent, an antimetabolites, retinal, tretinoin, isotretinoin, etretinate, acitretin, arotinoid, β-carotene, calcipotriene, anthralin, psoralen, 5-methoxypsoralen, trioxsalen, coal tar, masoprocol, and any pharmaceutically acceptable prodrug or derivative thereof.

39. The method of claim 36, wherein the compound is an immunosuppressive agent or procedure.

40. The method of claim 39, wherein the immunosuppressive agent or procedure is selected form the group consisting of cyclosporine, tacrolimus, azathioprine, mycophenolate, methotrexate, an immunoglobulin, a monoclonal antibody, an Rh(D) immune globulin, methoxsalen, thalidomide, radiation, or any pharmaceutically acceptable prodrug or derivative thereof.

41. The method of claim 36, wherein the compound is an antihistamine agent.

42. The method of claim 41, wherein the antihistamine agent is selected from the group consisting of carbinoxamine, clemastine, diphenhydramine, dimenhydrinate, pyrilamine, tripelennamine, chlorpheniramine, brompheniramine, hydrazine, cyclizine, meclizine, promethazine, acrivastine, cetirizine, astemizole, levocabastine, loratadine and terfenadine, or any pharmaceutically acceptable prodrug or derivative thereof.

43. A method for treating an immune-related disorder in a host comprising administration of an isolated fusion protein, wherein the isolated fusion protein comprises:
(a) a membrane-translocating sequence comprising at least eight consecutive residues of SEQ ID NO: 1 (Ala-Ala-Val-Leu-Leu-Pro-Val-Leu-Leu-Ala-Ala-Pro), and
(b) an IκB protein.

44. The method of claim 43, wherein the membrane-translocating sequence comprises at least nine consecutive residues of SEQ ID NO: 1.

45. The method of claim 43, wherein the membrane-translocating sequence comprises at least ten consecutive residues of SEQ ID NO: 1.

46. The method of claim 43, wherein the membrane-translocating sequence comprises at least eleven consecutive residues of SEQ ID NO: 1.

47. The method of claim 43, wherein the membrane-translocating sequence comprises at least twelve consecutive residues of SEQ ID NO: 1.

48. The method of claim 43, wherein the IκB protein comprises an IκBα protein.

49. The method of claim 43, wherein the IκB protein comprises an IκBβ protein.

50. The method of claim 43, wherein the IκB protein comprises an IκBεε protein.

51. The method of claim 43, wherein the IκB protein comprises a complex formed by two or more IκB proteins.

52. The method of claim 43, wherein the isolated fusion protein further comprises a tag amino acid sequence or protein.

53. The method of claim 43, wherein the tag amino acid sequence is selected from the group consisting of poly-arginine, poly-histidine, calmodulin-binding peptide (SEQ ID NO. 16), cellulose-binding domain, protein disulfide isomerase I (DsbA), c-myc (SEQ ID. NO. 12), glutathione S-transferase, a FLAG sequence (SEQ ID NO. 10), natural histidine tag (HAT; SEQ ID NO. 14), maltose-binding protein, transcript termination anti-termination factor (NusA), Staphylcoccal protein A, Staphylcoccal protein G, S-RNAase tag (SEQ ID NO. 13), streptavidin binding peptide (SEQ ID NO. 17), Strep-tag (SEQ ID. NO. 11), chitin-binding domain (SEQ ID NO. 18) and thioredoxin or any combination thereof.

54. The method of claim 43, wherein the isolated fusion protein further comprises an antibody.

55. The method of claim 43, wherein the immune response is associated with at least one of an allergy, asthma, contact dermatitis, delayed-type hypersensitivity, a wound-healing, allergic rhinitis, food hypersensitivity, ectopic dermatitis, imflammatory bowel disease, an immunologic disease of the lung, eosinophilic pneumonias, idiopathic pulmonary fibrosis, hypersensitivity pneumonitis, an autoimmune or immune-mediated skin disease, a bullous skin disease, erythemia multiforme, psoriasis, gluten-sensitive enteropathy, Whipple's disease, systemic lupus erythematisis, rheumatoid arthritis, osteoarthritis, juvenile chronic arthritis, ankylosing spondlylitis, systemic sclerosis, an idiopathic inflammatory myopathy, Sjögren's disease, pleuritis, sarcoidosis, amyloidisis, autoimmune hemolytic anemia, autoimmune thrombocytopenia, thyroiditis, diabetes mellitus, immune-mediated renal disease, myasthenia gravis, a demylenating disease of the central or peripheral nervous system, idiopathic demylenating polyneuropathy, Guillain-Barre syndrome, a chonic inflammatory demyelinating polyneuropathy, a hepotobiliary disease, an infectious or autoimmune chronic active hepatitis, primary biliary cirrhosis, granulomatous hepatitis, sclerlosing cholangitis, Graves disease, a transplantation-associated disease, a graft rejection, and graft-versus-host disease.

56. The method of claim 43, wherein the inflammatory response is caused by exposure to Mycoplasma tuberculosis.

57. The method of claim 43, wherein the fusion protein is administered therapeutically.

58. The method of claim 43, wherein the fusion protein is administered prophylactically.

59. The method of claim 43, wherein the isolated fusion protein is administered by one or more of the following routes selected from the group consisting of intravenous, intradermal, subcutaneous, oral, inhalation, deep lung inhalation, transdermal, transmucosal, vaginal and rectal administration.

60. The method of claim 43, wherein the isolated fusion protein is administered as a liposome.

61. The method of claim 43, wherein the isolated fusion protein is administered as an aerosol.

62. The method of claim 43, wherein the infusion protein is administered in combination with a compound used to treat or prevent any immune-related disorder.

63. The method of claim 62, wherein the compound is an anti-inflammatory agent.

64. The method of claim 63, wherein the anti-inflammatory agent is selected from the group consisting of aspirin, diflunisal, mesalamine, salicylsalicylic acid, sodium thiosalicylate, choline salicylate, magnesium salicylate, olsalazine, sulfasalazine, indomethacin, suldinac, etodolac, mefenamate, meclofenamate, flufenamate, tolfenamate, etofenamate, tolmetin, ketorolac, diclofenac, ibuprofen, naproxen, fenoprofen, ketoprofen, flurbiprofen, oxaprozin, piroxicam, meloxicam, nabumetone, apazone, nimesulide, zileuton, gold salts, colchicine, allopurinol, beclomethasone, budesonide, flunisolide, triamcinolone, prednisone, cromolyn, nedocromil, albuterol, bitolterol, pirbuterol, salmeterol, terbutaline, theophylline and other methylxanthines, metaproterenol, systemic glucocorticoids, antibiotics, antiparasitic agents, antiprotozoal agents, antimalarial agents, isoniazid, rifampin, ethambutol, antifungal agents, antiviral agents, alkylating agent, an antimetabolites, retinal, tretinoin, isotretinoin, etretinate, acitretin, arotinoid, β-carotene, calcipotriene, anthralin, psoralen, 5-methoxypsoralen, trioxsalen, coal tar, masoprocol, and any pharmaceutically acceptable prodrug or derivative thereof.

65. The method of claim 62, wherein the compound is an immunosuppressive agent or procedure.

66. The method of claim 65, wherein the immunosuppressive agent or procedure is selected form the group consisting of cyclosporine, tacrolimus, azathioprine, mycophenolate, methotrexate, an immunoglobulin, a monoclonal antibody, an Rh(D) immune globulin, methoxsalen, thalidomide, radiation, or any pharmaceutically acceptable prodrug or derivative thereof.

67. The method of claim 62, wherein the compound is an antihistamine agent.

68. The method of claim 67, wherein the antihistamine agent is selected from the group consisting of carbinoxamine, clemastine, diphenhydramine, dimenhydrinate, pyrilamine, tripelennamine, chlorpheniramine, brompheniramine, hydrazine, cyclizine, meclizine, promethazine, acrivastine, cetirizine, astemizole, levocabastine, loratadine and terfenadine, or any pharmaceutically acceptable prodrug or derivative thereof.

69. A method for treating or preventing an apoptosis-related disorder in a host comprising administration of an isolated fusion protein, wherein the isolated fusion protein comprises:
(a) a membrane-translocating sequence comprising at least eight consecutive residues of SEQ ID NO: 1 (Ala-Ala-Val-Leu-Leu-Pro-Val-Leu-Leu-Ala-Ala-Pro), and
(b) an IκB protein.

70. The method of claim 69, wherein the membrane-translocating sequence comprises at least nine consecutive residues of SEQ ID NO: 1.

71. The method of claim 69, wherein the membrane-translocating sequence comprises at least ten consecutive residues of SEQ ID NO: 1.

72. The method of claim 69, wherein the membrane-translocating sequence comprises at least eleven consecutive residues of SEQ ID NO: 1.

73. The method of claim 69, wherein the membrane-translocating sequence comprises at least twelve consecutive residues of SEQ ID NO: 1.

74. The method of claim 69, wherein the IκB protein comprises an IκBα protein.

75. The method of claim 69, wherein the IκB protein comprises an IκBβ protein.

76. The method of claim 69, wherein the IκB protein comprises an IκBεε protein.

77. The method of claim 69, wherein the IκB protein comprises a complex formed by two or more IκB proteins.

78. The method of claim 69, wherein the isolated fusion protein further comprises a tag amino acid sequence or protein.

79. The method of claim 78, wherein the tag amino acid sequence is selected from the group consisting of poly-arginine, poly-histidine, calmodulin-binding peptide (SEQ ID NO. 16), cellulose-binding domain, protein disulfide isomerase I (DsbA), c-myc (SEQ ID. NO. 12), glutathione S-transferase, a FLAG sequence (SEQ ID NO. 10), natural histidine tag (HAT; SEQ ID NO. 14), maltose-binding protein, transcript termination anti-termination factor (NusA), Staphylcoccal protein A, Staphylcoccal protein G, S-RNAase tag (SEQ ID NO. 13), streptavidin binding peptide (SEQ ID NO. 17), Strep-tag (SEQ ID. NO. 11), chitin-binding domain (SEQ ID NO. 18) and thioredoxin or any combination thereof.

80. The method of claim 69, wherein the isolated fusion protein further comprises an antibody.

81. The method of claim 69, wherein the apoptosis-related disease is cancer.

82. The method of claim 69, wherein the fusion protein is administered therapeutically.

83. The method of claim 69, wherein the fusion protein is administered prophylactically.

84. The method of claim 69, wherein the isolated fusion protein is administered by one or more of the following routes selected from the group consisting of intravenous, intradermal, subcutaneous, oral, inhalation, deep lung inhalation, transdermal, transmucosal, vaginal and rectal administration.

85. The method of claim 69, wherein the isolated fusion protein is administered as a liposome.

86. The method of claim 69, wherein the isolated fusion protein is administered as an aerosol.

87. An animal model to test the effects of an isolated fusion protein on an inflammatory response comprising:
(a) injecting an animal host with said fusion protein, wherein the animal host expresses a reporter gene whose expression is mediated by a NF-κB-dependent process such that inflammation may induce the production of the reporter gene product;
(b) stimulating inflammation;
(c) visualizing and quantifying the reporter gene product; and,
(d) comparing the amount of reporter gene product quantified in an animal host injected with said fusion protein with that of a control animal.

88. The animal model of claim 87, wherein the isolated fusion protein comprises:
(a) a membrane-translocating sequence comprising at least eight consecutive residues of SEQ ID NO: 1 (Ala-Ala-Val-Leu-Leu-Pro-Val-Leu-Leu-Ala-Ala-Pro), and
(b) an I κ B protein.
